(19) United States
(12) Patent Application Publication
(10) Pub. No.: US 2023/0024082 A1
(43) Pub. Date: Jan. 26, 2023

(54) TUBULAR BIOREACTOR

(71) Applicant: Jonathan L. Gal, Provo, UT (US)

(72) Inventor: Jonathan L. Gal, Provo, UT (US)

(21) Appl. No.: 17/813,307

(22) Filed: Jul. 18, 2022

Related U.S. Application Data
(60) Provisional application No. 63/222,641, filed on Jul. 16, 2021.

(51) Int. Cl. C12M 1/00; C12M 1/12

(52) U.S. Cl. CPC C12M 21/02; C12M 23/06

(57) ABSTRACT

A closed tubular bioreactor with clear corrugated or noncircular tubes having a constant cross-sectional area over their length, resulting in increased production.

### CROSS REFERENCE TO RELATED APPLICATIONS

Priority is claimed from U.S. Provisional Patent Application 63/222641, filed 16 Jul. 2021, which is hereby incorporated by reference

### BACKGROUND

Photosynthetic microorganisms can be used as a feedstock for many commercial and industrial applications: biofuels, human nutrition, feed for fish and farm animals, CO₂ sequestration, air purification, soil amendments and fertilizer, human cosmetics, color dyes, textiles, and bioplastics (Stanley, J. G. and Jones, J. B. 1976; Bennemann, J. R. 1979; Kumar, K. et al. 2011; Priyadarshani, I. and Rath, B. 2012; Rasala, B. A. and Mayfield, S. P. 2015; Christaki, E et al. 2017; Poonam, S. and Nivedita, S. 2017; Adeniyi, O. M. et al. 2018; Liu, J. et al. 2020; Arora, K. et al. 2021; Chong et al. 2021). The United States Department of Energy investigated photosynthetic microorganisms as a feedstock for biofuels during the 1980s and 1990s (Sheehan et al. 1996). Several companies are currently marketing such products (Cyanotech (https://www.cyanotech.com); TrueAlge (https://truealgae.com); Algeternal (https://algeternal.com)) (Tacon and Metian 2008). During 2019, the US National Renewable Energy Laboratory published a review of the state of the art for photosynthetic algae production (Clippinger and Davis 2019).

There are three steps in any industrial application of photosynthetic microorganisms: (1) Production, (2) Harvest and Processing, (3) Marketing and Delivery. This disclosure is focused exclusively on the production step, which is common to all the many applications listed above. Thus, the present invention could have a broad impact on all the applications mentioned above.

Production technology has historically been focused on the open pond system, also known as the ‘raceway’, and some still use that technique (Cyanotech http://www.cyanotech.com/). More recently, closed production systems—i.e. photobioreactors (PBRs)—have been built using clear, cylindrical piping (Phytobloom https://www.linkedin.com/company/phytobloom/). These are generally referred to as ‘tubular photobioreactors’ (Photobioreactor—Wikipedia https://en.wikipedia.org/wiki/Photobioreactor). Closed systems are more costly to build, but they offer advantages such as protection against contamination, control of culture conditions, and increased production rates (Photobioreactor—Wikipedia https://en.wikipedia.org/wiki/Photobioreactor).

Different variables can impact the production rate of a photobioreactor. Temperature, light, pH, salinity, nutrients, and gas exchange can all be varied. Each variable can distinctly impact culture growth (Hoseini et al. 2014; Kendirlioglu et al. 2017; Soni et al 2017, 2019; Srinivasan and Illanjiam 2021a, 2021b; Tayebati et al. 2020; Uslu et al. 2009; Wang et al. 2007). Finally, light distribution and management can also impact growth rates.

Cultures of photosynthetic algae have been observed to experience growth inhibition by the shadow effect (Frontasyeva et al. 2009; Olaizola et al. 1990; Soni et al. 2019). This occurs when the cell density is high enough that the cells shade each other, thus blocking each other's access to needed light. At particularly high concentrations, light cannot penetrate through even the surface layer to irradiate other cells deeper in the culture (González-Camejo et al. 2019; Shigesada and Okubo 1981).

### SUMMARY

An aspect is a tubular photobioreactor with a photo stage where light is captured for growth of algae. Referring to FIG. 6A, the photo stage 100 has one or more corrugated clear tubes 102 (FIG. 6B) through which a suspension of algae 104 or any suitable photosynthetic organism is continuously circulated.

The clear tubes having a center axis 106 and a corrugated cross-section. The corrugated cross-section defines a cross-section surface area, the cross-section surface area remaining constant along the center axis. The configuration of the corrugated cross-section may remain the same, or may vary as long the area remains constant, and the tube my have transitions between different cross-sections. An example, would be a transition from a circular end to a corrugated cross-section.

The walls 110 of corrugated clear tubes have a light transmittance such that the external surface area of the wall is a photo surface area (PSA) where external light is transmitted through the tube wall to the algae suspension. The material of the corrugated tubes may be of any suitable clear material, such as plastic or glass.

The clear tubes 102 can be straight or curved, i.e. the center axis may follow a straight 106 or curved path 106A. Along the center axis, the corrugated cross-section may be rotated around the axis to impart a helical or twisted structure to the clear tube (See dotted lines 130 in FIG. 6A). Alternately there may be now such rotation.

The corrugated cross-section may be defined by any suitable means, such as defined by a closed path 120 traveling around the center axis between two radii 121, 122 extending from the center axis. (FIG. 6B) The goal is to increase the photo surface area, which may mitigate against complex highly convoluted shapes with structure that would excessively block or shade surfaces.

To achieve a practical increase in production over conventional circular tubes, it is believed that the relative photon surface area of a corrugated tube should be greater than 10 percent, which is the percent increase of the photon surface area of a tube when compared with the photon surface area of a tube with a circular cross-section of the same area as the cross section area of the corrugated tube.

A suitable corrugated cross-section is defined by dividing a circle centered on the center axis into alternate pie-shaped sectors, with adjacent sectors having a different radius, as illustrated in FIG. 1B.

Another aspect is a prototypic tube with enhanced surface area, referred to herein as the AlgaTube™ and its ability to reduce the shadow effect and, thereby, improve the rate of photosynthetic production, when compared to the standard, cylindrical tubes that are widely used as state of the art in tubular photobioreactors. The results presented herein could help to improve the economics of any industrial process that utilizes photosynthetic microorganisms such as cyanobacteria and algae, including the ten applications listed above.

### BRIEF DESCRIPTION OF DRAWINGS

FIG. 1A and FIG. 1B represent the cross sections of the two tubes in the form of flasks. The control flask (FIG. 1A) is on the left, and the AlgaTube prototype flask (FIG. 1B) is on the right. They have identical cross-sectional areas, but the sidewalls of the two flasks were different. These diagrams are intended for illustrative purposes only. They are not presented to scale, nor are they intended for manufacturing or engineering purposes. The key geometric measures of the respective flasks are presented in Table 1.

FIG. 2 is a photographic view of a test tubular photobioreactor to measure performance of flasks as in FIG. 1A and FIG. 1B. The control flask was on the left, and the prototype flask was on the right. The two light panels were affixed to the rear wail of the incubator. The incandescent light was attached to the door and centered. Temperature was 34° C. Lights were on 24/7. Air bubblers ran 24/7. Pure CO₂ was dosed as needed, using an automated pH controller set for 9.0±0.2. Magnets were affixed to the sides; and caps were on top. Average incident Light Intensity (iPPFD_{avg}) was 55 μmol/m²/s at the perimeter of the flasks.

FIG. 3A and FIG. 3B-FIG. 3A is a scatter plot of the daily Dry Weight measurements for each of the four experimental replicates. Each point represents a specific day for a specific replicate. The prototype data points are represented by solid lines and black triangles. The control data points are represented by dashed lines and black circles. FIG. 3B plots the average of the Dry Weight data for the four replicates. As in FIG. 3A, the prototype data was represented by solid lines and black triangles; the control data was represented by dashed lines and black circles. Δ_{DW10}=+34% (p=0.02, Wilcoxon Rank Sum Test).

FIG. 4A and FIG. 4B. FIG. 4A is a scatter plot of the Cumulative Production data for each of the four experimental replicates. Each point represents the Cumulative Production data on a specific day for a specific replicate. The prototype data points are represented by solid lines and black triangles (▴), and the control data points are represented by dashed lines and black circles. FIG. 4B plots the average of the Dry Weight data for the four replicates. As in FIG. 4A, the prototype data points are represented by solid lines and black triangles, and the control data points are represented by dashed lines and black circles . Δ_{CP10}=+41% (p=0.02, Wilcoxon Rank Sum Test).

FIG. 5. The Average Daily Production data were, themselves, averaged together longitudinally, for each of three different time periods within the 10-day experiment. To compare the Average Daily Production rates for different time periods, the figures are presented in bar chart format, along with their respective Delta (Δ_{ADp}) values. For reference, the average culture concentration during each of those time periods was shown below the x axis labels. The bars representing the control flask are white, and the bars representing the prototype flask are shaded gray.

FIG. 6A and FIG. 6B are schematics illustrating an exemplary embodiment of the invention.

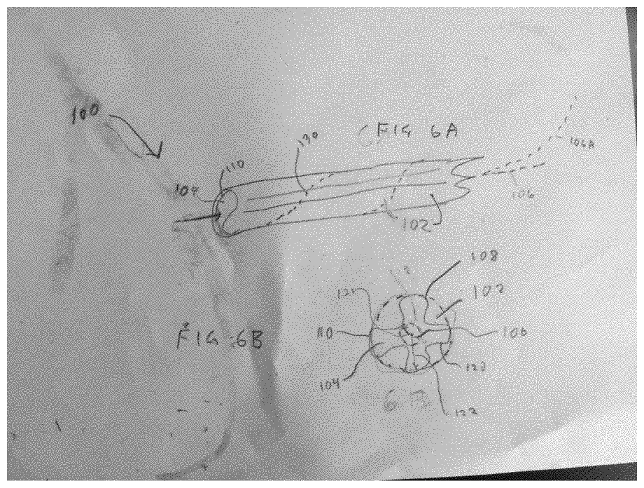

### DETAILED DESCRIPTION

The cross-sectional area of an AlgaTube is chosen depending upon its use, for example, the construction, capacity, operation and function of the bioreactor. Because of the non-circular nature of the cross-section of an AlgaTube, there is no single diameter, but a diameter measure comparable to the diameter of circular tubes and be made as an Equivalent Diameter. Equivalent diameter can apply to any tube, and is the the diameter of a circular cross-section that has the same area as the cross-section of the tube. Accordingly in a bioreactor design, AlgaTubes may be substituted for circular tubes having the same equivalent diameter. The application determines the equivalent diameter of an AlgaTube. It may be very small, in the micrometer or nanometer range, or very large, on the order of 100 meters is diameter.

An AlgaTube may be of any suitable material or mixture of materials of construction, including but not limited to, glasses, plastics, graphene, or fiberglass.

Algatubes may be modified with ports or holes to accommodate scientific instruments, probes, and like, for monitoring the culture conditions.

Various adaptors, connectors, curved conduits, and other kinds of fittings may be required to connect components together.

### EXAMPLE

1. AlgaTube Evaluation

An AlgaTube was evaluated for growth of photosynthetic microorganisms in a controlled, 10-day batch trial at low light intensity. Two cultures (control and prototype) were grown in identical conditions.

A laboratory scale prototype of a AlgaTube™ was evaluated inside a closed incubator space at 34° C. with an average light intensity of 55 μmol/m²/s LED light, consisting mostly of red and blue wavelengths. Biomass concentration was assessed daily by converting 590 nm Percent Light Transmission (% T₅₉₀) to Dry Weight (DW in g/l) using a standard curve that correlated dry weight of Spirulina with % T₅₉₀. Cumulative Production on each day was calculated by subtracting the initial concentration of 0.25 g/l from each day's Dry Weight measurements. Ten-day Cumulative Production was 41% higher in the prototype, and this was determined to be statistically significant (p-value=0.03) using the Wilcoxon Rank Sum Test.

2. Materials and Methods

2.1. Culture Vessels

A prototype flask of the AlgaTube™ was made by 3-D printing, using DMS Somos® Watershed XC 11122 as the resin and Stereolithography (SLA) as the printing method. The control flask was also made using the same 3D printing process and material. Due to the relatively low heat deflection temperature of this resin, each container was supported with a stainless steel hose clamp to prevent warping and deformation. The hose clamps blocked approximately 7% of incident PAR on each flask. The light transmittance properties of the Watershed XC 11122 resin are similar to clear acrylic plastic with % T in the range 70% -85%, depending on the wavelength (Covestro Additive Manufacturing https://am.covestro.com/en_US/home.html).

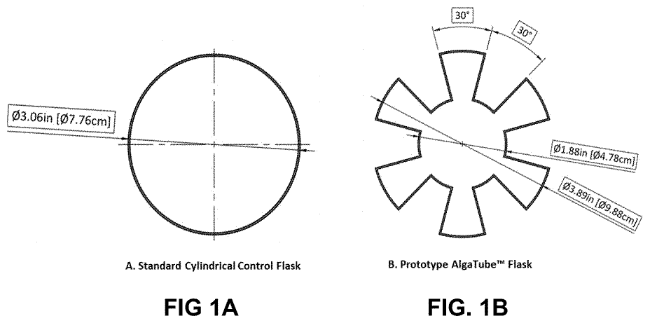

The term “Photon Surface Area” is used to describe the surface area of the sidewalls of the vessels (not including the top and bottom of the vessel), and the term “Cross Section Surface Area” is used to describe the cross-sectional area of the vessels, the area across which gas exchange and evaporation occur. The shape of the sidewalls, and thus the Photon Surface Area, was the only variable that differed between the two vessels (FIG. 1). Cross Section Surface Area, Height, and Volume were identical (Table 1).

TABLE 1
Key geometric measures of the two flasks

| Geometric measure | Control | Prototype | Units |
|---|---|---|---|
| Capacity Volume^{a} | 1,441 | 1,441 | ml |
| Photon Surface Area^{b} | 743 | 1,641 | cm² |
| Relative Photon Surface Area^{c} | 100% | 220% | % of control |
| Cross Section Surface Area^{d} | 47 | 47 | cm² |
| PSA/Volume Ratio^{e} | 0.52 | 1.13 | cm⁻¹ |
| Relative PSA/Volume Ratio^{f} | 100% | 220% | % of control |

Six key geometric measures for each of the two flasks used in this experiment.
^{a}Capacity Volume: total volume of the flask when full to brim.
^{b}Photon Surface Area: total surface area of the sidewalls of the flask.
^{c}Relative Photon Surface Area: expressed as %, with control = 100%.
^{d}Cross Section Surface Area: area across which gas exchange and evaporation occur.
^{e}PSA/Volume Ratio: ratio of Photon Surface Area to Volume of each flask.
^{f}Relative PSA/Volume Ratio: expressed as % with control = 100%.

2.2. Culture Species and Medium

A. Platensis (Spirulina) was chosen and purchased from Algae Research Supply in California, USA (Algae Research Supply). Modified Schlosser Medium (da Silva et al. 2016) was prepared using deionized water.

2.3

(Empty)

2.4. Light

Two YOHAYOH LED Light Panels [45 W, 50-60 Hz, 300 mA, 110V, 30.99 cm (12.2 in)×30.99 cm(12.2 in)×4.06 cm(1.6 in)] were affixed to the inner, rear side of the incubator. The light panels emitted narrow spectral peaks at 460 nm (blue) and 630 nm (red). To enhance stimulation of P700, a photoreceptor that absorbs 700 nm light, a 15-Watt incandescent bulb (General Electric) was added (Webber et al. 2001; Thorseth 2015). The lights were on 24 hr/day (Prates et al. 2018) during the ten-day period.

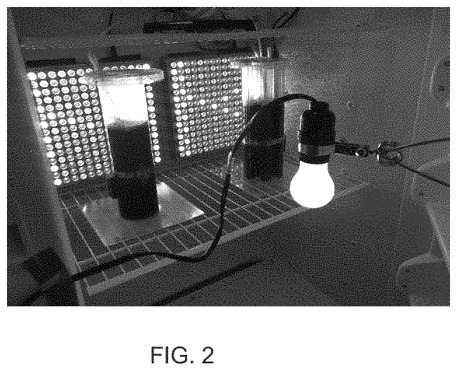

In FIG. 2, the incubator door was open. During most of the experiment, however, the door was closed. The inside walls of the closed incubator space were white, ensuring a high degree of diffuse reflection of PAR (Warman and Mayhew 1979). Using an enhanced silicon photodiode assembly (LI-190R Quantum Sensor), the light intensity was measured at 24 spot locations around the perimeter of the flasks. Spot readings were within a range of 22-105 μmol/m²/s. With the door closed, the average light intensity around the perimeter of the flasks was 55 μmol/m²/s (iPPFD_{avg}).

2.5. Magnetic Fields

Identical magnets were attached to the outside of each vessel (Li et al. 2007; Deamici et al. 2016), such that the two cultures were exposed to a magnetic field of approximately 300 mT (milliTesla) each (FIG. 2). The magnets (K&J Magnetics) were Neodymium Grade N52 with a Brₘₐₓ rating of 14,800 Gauss and a BHₘₐₓ rating of 52 MGOe. They were encapsulated in black rubber (by the manufacturer) to prevent shattering of the brittle material.

2.6. Temperature and Humidity

The temperature was 34 degrees C. throughout the experiment. This temperature is within a previously published range of preferred temperature for Spirulina (Soni et al. 2019; Oliveira et al. 1999). The incubator was VWR Model #3733A (115V, 60 Hz, 6.2 A, 1 PH, manufactured in the USA). Daily, deionized water from the BYU Life Sciences Building was added to replace evaporative water loss. The Relative Humidity inside the incubator tended to be in the 10% -15% range, but it did occasionally fluctuate outside that range during the passage of meteorological storm systems through Provo, Utah, USA. Fluctuations in humidity, while not ideal, impacted both the control and the prototype identically. Thus, comparisons between the two are still valid as apples-to-apples comparisons, irrespective of the fluctuations in humidity.

2.7. Gases and pH

Pure gaseous CO₂ was supplied from a pressurized tank through a regulator (FZone Model #FZ-2020PRO), a pH controller (Milwaukee MC122Pro), and a sparger stone (CR Brewbeer, 0.5 um Diffusion Stones). The pH controller was set to 9.0±0.2 (Ismaiel et al. 2016). To reduce buildup of gaseous Oxygen (Kazbar et al. 2019), ambient air was bubbled through both cultures using a HITOP Aquarium Air Pump (Haisen).

2.8. Daily Data Collection

Prior to daily sampling, each culture was mixed manually to promote homogeneity. The cultures were tested for Dissolved Oxygen (RCYAGO Portable Dissolved Oxygen, Model #DO9100), pH (Milwaukee MC122Pro), Total Dissolved Solids and Electrical Conductivity (Vivosun TDS/EC Meter), and Light Transmittance and Absorption (Biolog Turbidimeter, Model #21907, 590 nm). Dry Weight (g/l) was interpolated from % T₅₉₀ using a standard curve.

The organisms formed clumps of spiral filaments, which was a source of error during the OD₅₉₀ readings. Clumps caused some minor, but noticeable, drift in the analog turbidity readings as they circulated in the culture samples. To reduce the margin of error associated with this imperfect homogeneity, the turbidity meter was observed for a full minute for each reading. Time weighted averages of the % T₅₉₀ and ABS₅₉₀ were recorded for each measurement, rather than single spot readings.

2.9. Calculations

2.9.1. Delta (Δ)

To measure performance differences between the two vessels, Delta (Δ) was calculated using the control datasets and the prototype datasets. Delta (Δ) is expressed in percentage terms, relative to the control. It is not specific to any particular metric. A Delta (Δ) can be calculated for any metric and any time period and labeled accordingly. For example, the difference between the prototype culture concentration and the control culture concentration (DW in g/l) on day #5 would be labeled Δ_{Dw5}. The difference in Cumulative Production (CP in g/l) on day #8 would be labeled Δ_{CP8}. And the difference in Average Daily Production (ADP in g/l/day) during the first seven days would be labeled Δ_{ADP1-7}. The calculation of Delta (Δ) for Dry Weight on day #9 is presented here.

Δ_{DW9}=100%×(1.71g/l−1.34 g/l)÷(1.34 g/l)=+28% (1)

2.9.2 Statistical Significance

The datasets were determined to follow non-normal distributions, which prevented the use of a standard t test. Therefore, a non-parametric test was used. Statistical significance was assessed by the Wilcoxon Rank Sum Test, a.k.a. the Mann-Whitney U Test (Corder and Foreman 2014). The test result was a p-value. When the test produced a p-value≤0.05, then statistical significance had been achieved.

3. Results and Discussion

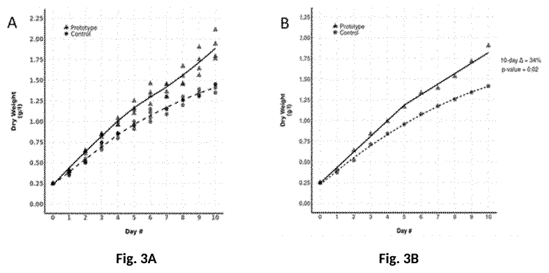

A Dry Weight (DW in g/l) dataset consisting of four independent replicates was recorded and plotted in FIG. 3. Both the control and prototype Dry Weight data exhibited non-linear trends, and the two flasks each produced different shaped growth trends. A quadratic model was fit to the control data. A non-constant variance linear spline regression model was fit to the prototype data. The prototype data points from the four independent replicates also showed increasing variance as the day number increased, a result that was not observed in the control flask. This increasing variance in the prototype data was likely caused by imperfect mixing of culture contents in the prototype, compared to the more thorough mixing of the cylindrical control. The corrugated sidewalls of the prototype partially interfered with culture mixing and culture homogeneity, especially as the cultures thickened. Nevertheless, the averages still showed distinct growth differences in favor of the prototype. Δ_{DW10} was +34%, and it was statistically significant (p=0.02).

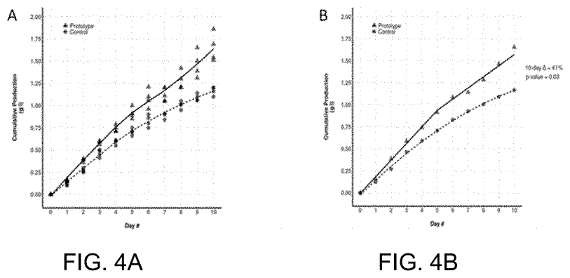

Next, Cumulative Production was calculated by subtracting the initial concentration of biomass (0.25 g/l) from the total biomass on each day. A Cumulative Production dataset was recorded and plotted in FIG. 4. The percentage difference between the average Cumulative Production results was calculated (Δ_{CP10}).

10-day Cumulative Production (control)=1.42 g/l−0.25 g/l=1.17 g/l (2)

10-day Cumulative Production (prototype)=1.90 g/l−0.25 g/l=1.65 g/l (3)

Δ_{CP10}=100%×(1.65g/l−1.17g/l)÷(1.17g/l)=+41% (4)

Finally, Daily Production was calculated by subtracting the previous day's biomass concentration from each day's measurement. The resulting dataset focuses attention on the amount of Dry Weight produced per day, rather than the cumulative total. The Maximum Daily Production (MDP) in the prototype dataset, taken from amongst all four replicates, was 0.36 g/l/day. That was 50% greater than the comparable figure for the control, which was 0.24 g/l/day. The four replicates were averaged together, and the Average Daily Production dataset was then parsed into three separate time periods: (1) the full 10 days; (2) the first seven days; and (3) the last three days. A longitudinal average was calculated for each time period. Between the first seven days and the last three days, Average Daily Production (ADP) in the control flask dropped by 38% from 0.13 g/l/day down to 0.08 g/l/day; and this caused a dramatic increase in the Delta (Δ) during the final three days. At +113%, A_{ADP8}-₁₀ was nearly 5 times higher than Δ_{ADP1-7}, which was +23%.

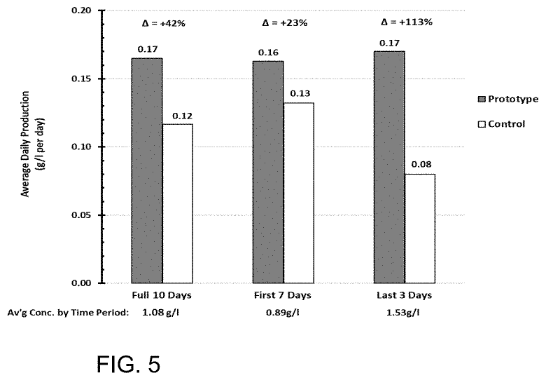

It is clear from this time parsed data analysis that the biomass growth in the control flask was negatively impacted by the shadow effect during the final three days of the trials, but the prototype did not experience the same negative impact during that same time period (FIG. 5). When viewed alongside the Delta (Δ) values noted in FIG. 3 and FIG. 4, this observation confirms that the enhanced surface area of the prototype did, in fact, reduce the shadow effect and enable the culture to maintain a higher rate of growth for a longer time period, when compared to the cylindrical control flask.

While the difference in PSA between the two cultures was +120%, the difference in CP was +41%. The continuous mixing of the cultures by the air bubblers was probably the reason that the growth advantage of the prototype was not as great as the difference in PSA, particularly at lower concentrations. At those lower concentrations, the mixing of the cultures partially overcame the shadow effect; but as the cultures became thicker, then mixing became less effective at preventing the shadow effect.

When Spirulina cultures are thick, they do not mix as well. The relatively large and spiral shaped filaments of Spirulina are likely to bump into each other, like bumper cars, or even to hook onto each other mechanically, both of which create something like a ‘traffic jam’ of filaments inside a culture vessel. There may also be biochemical reasons for the clumping of Spirulina filaments in culture. Filaments that are stuck inside of these ‘traffic jams’ absorb less light, because they are shaded by nearby filaments.

Another industrial bioprocess that may benefit from corrugated clear tubesworis the use of photobioreactors as disclosed for photosynthetic waste water remediation. By speeding the rate of growth of the algae in such applications, the AlgaTube™ may improve the efficiency of this type of industrial bioprocess, as well.

It should be noted that the light intensity inside the incubator was considerably different with the door open, compared to the door being closed. With the door open, the iPPFD_{avg} was measured to be 29 μmol/m²/s, which was 55% lower than the iPPFD_{avg} of 55 μmol/m²/s when the door was closed. This data comparison is noteworthy for the field of photobioreactor design, because it shows that light containment is an important consideration in photobioreactor design. Without containment, a large percentage of PAR can be lost to the surrounding environment without ever being used for biomass growth.

4. Conclusions

In a 10-day controlled study at laboratory scale, the AlgaTube™ prototype with enhanced surface area increased the Cumulative Production of biomass by 41%, compared to a cylindrical control, in low light. The result was statistically significant (p=0.03). Comparative analysis of different time periods showed that the prototype improved the Average Daily Production by 113% during the final three days of the trial, when the cultures were at their thickest. With all other variables having been carefully controlled, the enhanced surface area of the AlgaTube™ prototype was determined to be the sole factor causing these improvements.

The invention has been described with reference to various specific and preferred embodiments and techniques. Nevertheless, it is understood that many variations and modifications may be made while remaining within the spirit and scope of the invention.

### REFERENCES

Chong, J. W. R., Khoo, K. S., Yew, G. Y., Leong, W. H., Lim, J. W., Lam, M. K., Ho, Y. C., Ng, H. S., Munawaroh, H. S. H., Show, P. L. 2021. Advances in production of bioplastics by microalgae using food waste hydrolysate and wastewater: A review. Bioresour. Technol. 342, 125947.

Clippinger, J. and Davis, R. 2019. Techno-Economic Analysis for the Production of Algal Biomass via Closed Photobioreactors: Future Cost Potential Evaluated across a Range of Cultivation System Designs. National Renewable Energy Laboratory, Golden, Colo. NREL/TP-5100-72716, https://www.nrel.gov/docs/fv19osti/7271.

Corder, G. W. and Foreman, D. I. Nonparametric Statistics: A Step-by-Step Approach, 2^{nd} Ed. 2014. John Wiley & Sons, Inc. Hoboken, N.J.

da Silva, M. F., Casazza, A. A., Ferrari, P. F., Perego, P., Bezerra, R. P., Converti, A., Porto, A. L. F., 2016. A new bioenergetic and thermodynamic approach to batch photoautotrophic growth of Arthrospira (Spirulina) platensis in different photobioreactors and under different light conditions. Bioresour. Technol. 207, 220-228.

Deamici, K. M., Costa, J. A. V., Santos, L. O., 2016. Magnetic fields as triggers of microalga growth: evaluation of its effect on Spirulina sp. Bioresour. Technol. 220, 62-67.

Frontasyeva, M. V., Pavlov, S. S., Mosulishvili, L., Kirkesali, E., Ginturi, E., and Kuchava, N., 2009. Accumulation of trace elements by biological matrix of Spirulina platensis. J. Soc. Ecol. Chem. and Eng. 16(3) 277-285.

González-Camejo, J., Viruela, A., Ruano, M. V., Barat, R., Seco, A., Ferrer, J., 2019. Dataset to assess the shadow effect of an outdoor microalgae culture. Data in Br. 25, 104143.

Hoseini, S. M., Almodares, A., Afsharzadeh, S., Shahriari, A. R., Montazeri, F. 2014. Growth response of Spirulina platensis PCC9108 to elevated CO₂ levels and flue gas. Biol. J. Microorg. 2(8), 29-36.

Ismaiel, M. M. S., El-Ayouty, Y. M., Piercey-Normore, M., 2016. Role of pH on antioxidants production by Spirulina (Arthrospira) platensis. Braz. J. of Microbiol. 47(2), 298-304.

Kazbar, A., Cogne, G., Urbain, B., Marec, H., Le-Gouic, B., Tallec, J., Takache, H., Ismail, A., Pruvost, J., 2019. Effect of dissolved oxygen concentration on microalgal culture in photobioreactors. Algal Res. 39, 101432.

Kendirlioglu, G., Cetin, A. K, 2017. Effect of Different Wavelengths of Light on Growth, Pigment Content, and Protein Amount on Chlorella Vulgaris. Fresenius Environ. Bull. 26(12A), 7974-7980.

Li, Z.-Y., Guo, S.-Y., Li. L., Cai, M.-Y., 2007. Effects of electromagnetic field on the batch cultivation and nutritional composition of Spirulina platensis in an air-lift photobioreactor. Bioresour. Technol. 98(3), 700-705.

Olaizola, M. and Duerr, E. O. 1990. Effects of light intensity and quality on the growth rate and photosynthetic pigment content of Spirulina platensis. J. Appl. Phycol. 2 97-104.

Oliveira, M. A. C. L de, Monteiro, M., Robbs, P. G., Leite, S. G. F., 1999. Growth and Chemical Composition of Spirulina Maxima and Spirulina Platensis Biomass at Different Temperatures. Aquac. Int. 7, 261-275.

Prates, D.daF., Radmann, E. M., Duarte, J. H., de Morais, M. G., Costa, J. A. V., 2018. Spirulina cultivated under different light emitting diodes: Enhanced cell growth and phycocyanin production. Bioresour. Technol. 256, 38-43.

Sheehan, J. Dunahay, T. G., Benemann, J. R., Roessler, P. G., Weissman, J. C., 1998. A Look Back at the U.S. Department of Energy's Aquatic Species Program—Biodiesel from Algae. National Renewable Energy Laboratory, Golden, CO, US Department of Energy, Close-out Report. https://www nrel.gov/docs/legosti/fy98/24190.pdf

Shigesada, N., and Okubo, A., 1981. Analysis of the self-shading effect on algal vertical distribution in natural waters. J. Math. Biol. 12, 311-326.

Soni, R. A., Sudhakar, K., Rana, R. S. 2017. Spirulina—From growth to nutritional product: A review. Trends Food Sci. Technol. 69(A), 157-171.

Soni, R. A., Sudhakar, K., Rana, R. S. 2019. Comparative study on the growth performance of Spirulina platensis on modifying culture medium. Energ. Rep. 5, 327-336.

Srinivasan, T. and Illanjiam, S., 2021a. Extraction and Purification of Phycocyanin and Their Radical-Scavenging Activity from Multi-Stress Spirulina Isolated from Marine Water. Appl. Ecol. Environ. Sci. 9(1), 73-75.

Srinivasan, T. and Illanjiam, S. 2021b. Optimization Studies of Multistress Spirulina Isolated from Marine Water. Appl. Ecol. Environ. Sci. 9(1), 76-78.

Tacon, A. G. J. and Metian, M. 2008. Global overview on the use of fish meal and fish oil in industrially compounded aquafeeds: Trends and future prospects. Aquac. 285(1-4), 146-158.

Tayebati, H., Shariati, F. P., Soltani, N., Tehrani, H. S. 2020. The effect of different light variables on spirulina growth and its component. Iran. Int. Conf. Chem. Eng. Congr. Exhib. 11, https://www.researchqate.net/profile/Hanieh-Tavebati/publication/345775863.

Thorseth, A. 2015. Spectral power distribution of a 25 W incandescent light bulb. Dep. Photonics Eng. Tech. Univ. of Den. Incandescent Light Bulb—Wikipedia.

Usiu, L. H., Oya, I., Sayin, S., Durrnaz, Y., Göksan, T., & Gökpinar, S. (2009). The Effect of Temperature on Protein and Amino Acid Composition of Spirulina platensis. Ege J. Fish. Aquat. Sci. 26(2), 139-142.

Wang, C. Y., Fu, C.-C., Liu, Y.-C., 2007. Effects of using light-emitting diodes on the cultivation of Spirulina platensis. Biochem. Eng. J. 37(1), 21-25.

Warman, P. R. and Mayhew, W. J., 1979. Effect of reflective surfaces on a greenhouse lettuce crop. Canada. https://www.osti.gov/etdeweb/biblio/8487983

Webber, A. N., Lubitz, W. 2001. P700: the primary electron donor of photosystem I. BBA Bioenerg. 1507(1-3), 61-79.

Weyer, K. M., Bush, D. R., Darzins, A., Willson, B. D. 2010. Theoretical Maximum Algal Oil Production. BioEnerg. Res. 3, 204-213.

### WEB REFERENCES

1. Covestro Additive Manufacturing; https://am.covestro.com; 2/3/22.

2. Cyanotech; http://www.cyanotech.com; 2/3/22.

3. TrueAlgae; http://www.truealgea.com; 2/3/22.

4. AlgEternal Technologies; http://www.algeternal.com/; 2/3/22.

5. Meticulous Research; https://www.meticulousresearch.com/product/spirulina-market-5070; 2/3/22.

6. Photobioreactor—Wikipedia; https://en.wikipedia.org/wiki/Photobioreactor; 2/3/22

7. Algae Research Supply; https://algaeresearchsupply.com/; 2/3/22.

8. Amazon.com: YOHAYOH LED Grow Lights: https:///www.amazon.com/YOHAYOH-Lights%EF%BC%8CGrow-Greenhous-Seedlings-Flowering/dp/B07SW3DWM4/ref=sr1 5 ?crid=13GSKLZK37ZQ4&keywords=YOHAYOH&qid=1643915314&sprefix=yohoyoh%2Caps%2C108&sr=8-5; 2/3/22.

What is claimed is:

1. A tubular photobioreactor comprising:
a photo stage having one or more corrugated clear tubes through which algae suspension is being continuously circulated,
the corrugated clear tubes having a center axis and a corrugated cross-section, the corrugated cross-section defining a cross-section surface area, the cross-section surface area remaining constant along the center axis,
the walls of corrugated clear tubes have a light transmittance such that surface area of the wall is a photon surface area where external light is transmitted through the wall to the algae suspension for algae growth.

2. A tubular photobioreactor comprising:
a photo stage having one or more corrugated clear tubes through which a suspension of photosynthetic organisms is being continuously circulated,
the corrugated clear tubes having a center axis and a corrugated cross-section, the corrugated cross-section defining a cross-section surface area, the cross-section surface area remaining constant along the center axis,
the walls of corrugated clear tubes have a light transmittance such that surface area of the wall is a photon surface area where external light is transmitted through the wall to the suspension for photosynthesis,
wherein the relative photon surface area is greater than 10 percent, when the corrugated clear tube is compared with a circular tube which has a circular cross-section of the same area as that of he corrugated clear cross section area.

3. A tubular photobioreactor as in claim 1 where the center axis follows a straight or curved path.

4. A tubular photobioreactor as in claim 1 where in the corrugated cross-section is rotated around the center axis along the center axis to form a helical-like construction of the tube.

5. A tubular photobioreactor as in claim 1 wherein the corrugated cross-section is defined by a closed path traveling around the center axis between two radii extending from the center axis.

6. A tubular photobioreactor as in claim 1 wherein the corrugated cross-section is defined by dividing circle into alternate pie-shaped sectors, with adjacent sectors having a different radius.

7. A tubular photobioreactor as in claim 1 wherein the corrugated clear tube is constructed of one or more chosen from glasses, plastics, graphene, and fiberglass.
